(19) United States
(12) Patent Application Publication
(10) Pub. No.: US 2022/0370443 A1
(43) Pub. Date: Nov. 24, 2022

(54) METHOD OF TREATING ALZHEIMER'S DISEASE

(71) Applicant: AMYRIAD PHARMA, INC., Los Angeles, CA (US)

(72) Inventor: Sharon L. ROGERS, Los Angeles, CA (US)

(21) Appl. No.: 17/748,451

(22) Filed: May 19, 2022

Related U.S. Application Data
(60) Provisional application No. 63/190,299, filed on May 19, 2021.
(60) Provisional application No. 63/192,398, filed on May 24, 2021.
(60) Provisional application No. 63/331,011, filed on Apr. 14, 2022.

(51) Int. Cl. A61K 31/497; A61K 31/445; A61K 31/13; A61P 25/28; A61K 9/20; A61K 45/06

(52) U.S. Cl. CPC A61K 31/497; A61K 31/445; A61K 31/13; A61P 25/28; A61K 9/20; A61K 45/06

(57) ABSTRACT

The present disclosure is directed to the treatment of Alzheimer's disease by administering 1′,3′-dihydro-2H-spiro[imidazo[1,2α]pyridine-3,2′-inden]-2-one orally at a daily dose of 180 mg as a single active agent or co-administered with donepezil hydrochloride and/or memantine hydrochloride.

### CROSS REFERENCE TO RELATED APPLICATIONS

This application claims the benefit of priority to U.S. Provisional Patent Application Ser. No. 63/190,299, filed on May 19, 2021, U.S. Provisional Patent Application Ser. No. 63/192,398, filed on May 24, 2021, and U.S. Provisional Patent Application Ser. No. 63/331,011, filed on Apr. 14, 2022, the contents of which are hereby incorporated by reference in their entireties.

### FIELD

The present disclosure describes administering 1′,3′-dihydro-2H-spiro[imidazo-[1,2α]pyridine-3,2′-inden]-2-one (“AD101”) to treat patients with Alzheimer's disease. In one aspect, AD101 is administered in an improved dosage regimen. In one aspect, AD101 is administered to patients receiving memantine hydrochloride therapy.

### BACKGROUND

Alzheimer's Disease (“AD”) is a neurodegenerative disorder for which there are only symptomatic treatments, with limited efficacy. It is predicted that the global prevalence of AD will quadruple by 2050 to over 100 million, at which time 1 in 85 people worldwide will be living with the disease. More than 40 percent of those cases will be in late stage AD requiring a high level of attention equivalent to nursing home care. AD starts with mild cognitive problems, such as memory loss ultimately progressing to the stage where independent living is not possible. The principal risk factors for developing AD is age; the likelihood of developing Alzheimer's doubles about every five years after age 65, and after age 85, the risk reaches nearly 50 percent. A family history also increases the risk of developing the disease, which may be due to genetics or environmental factors.

AD is the most common cause of dementia. The term dementia describes a syndrome characterized by dysmnesia, intellectual deterioration, personality changes and behavioral abnormalities. These symptoms result in social and occupational decline. Dementia can have multiple etiologies and pathophysiologies, and a range of drugs are being developed to treat this condition. Dementia of the Alzheimer type (“DAT”) is defined as a progressive, fatal neurodegenerative condition characterized by deterioration in cognition and memory, progressive impairment in the ability to carry out activities of daily living, and a number of neuropsychiatric and behavioral symptoms. DAT is the most common form of dementia among the elderly, and is expected to increase as the population ages.

There are currently four drugs approved for the treatment of cognitive symptoms of AD, classified in two groups:
1. cholinesterase inhibitors (ChEIs): donepezil, rivastigmine and galantamine. These drugs increase cholinergic transmission by inhibiting cholinesterases that hydrolyze it. Galantamine is used to treat mild to moderate AD, ARICEPT® (donepezil hydrochloride tablets) and rivastigmine are also indicated for AD, and may be used in patients with mild, moderate or severe disease.
2. NMDA (N-methyl-D-aspartic acid) receptor antagonists: memantine hydrochloride. This drug modulates the effects of pathologically elevated tonic levels of glutamate that may lead to neuronal dysfunction. NAMENDA® (memantine hydrochloride tablet) is indicated only for the treatment of patients with moderate to severe DAT.

None of these approved drugs represent a cure for the disease. In addition, non-selective ChEIs have notable undesirable side effects such as vomiting and diarrhea. Therefore, there is a need for other treatment options to mitigate the deterioration of cognitive impairment and global function (i.e. the overall ability of patients to function in their everyday activities) in patients with AD. Furthermore, although memantine hydrochloride is generally well-tolerated when administered to subjects with AD, it exhibits certain side effects that can be exacerbated and/or lead to treatment complications when given in combination with other AD drugs.

AD101 (previously reported as AD101, ST101 or ZTET1446) is a small molecule having the chemical name 1′,3′-dihydro-2H-spiro[imidazo[1,2α]pyridine-3,2′-inden]-2-one. AD101 and its preparation were first described in WO 2001/09131A1, the contents of which are incorporated herein by reference. The present disclosure provides a daily dose of AD101 which may be particularly effective in treating subjects with Alzheimer's Disease (AD), including subjects who are currently receiving a stable regimen of donepezil hydrochloride. Thus, AD101 administered orally at 180 mg once-daily (QD) provides symptomatic relief to AD subjects, including mitigation of cognitive impairment and global function in patients showing onset or development of AD and/or improving cognition and global function in treated subjects.

The present disclosure also provides a new combination therapy which may be particularly effective in treating subjects with AD. Thus, the present disclosure describes that memantine hydrochloride and AD101 can be safely co-administered. Surprisingly, AD101 can be administered at a daily dose up to at least 180 mg to subjects with AD who are taking a stable dose of memantine hydrochloride without any resulting significant side effects. It is also surprising that this favorable side effect profile is retained when AD101 is given at a daily dose up to at least 180 mg to subjects with AD who are taking a stable dose of memantine hydrochloride and a stable dose of donepezil hydrochloride.

The co-administration of AD101 and memantine hydrochloride may provide particular symptomatic relief to AD subjects (including subjects who are currently receiving a stable regimen of donepezil hydrochloride), including mitigation of cognitive impairment and global function in patients showing onset or development of Alzheimer's disease and/or improving cognition and global function in treated subjects. The combination therapy may be effective without concomitant significant safety issues. Therefore, the administration of AD101 (e.g. 180 mg AD101 QD) to subjects with AD currently treated with memantine hydrochloride and/or donepezil hydrochloride may provide particularly effective symptomatic relief without introducing significant drug-related safety concerns.

### SUMMARY

The present disclosure describes, in one aspect, that AD101 may be safely administered at a daily dose (QD) of 180 mg to subjects with Alzheimer's disease.

The present disclosure describes, in one aspect, that AD101 may be safely administered at a daily dose (QD) of 180 mg to subjects with dementia of the Alzheimer's type.

The present disclosure describes, in one aspect, that AD101 may be safely co-administered with memantine hydrochloride to subjects with Alzheimer's disease. In one embodiment of this aspect, AD101 may be administered at a daily dose (QD) of up to at least 180 mg. In a further embodiment of this aspect, the subject is also administered donepezil hydrochloride (e.g. including when AD101 is administered at a daily dose (QD) of up to at least 180 mg).

The present disclosure describes, in one aspect, that AD101 may be safely co-administered with memantine hydrochloride to subjects with dementia of the Alzheimer's type. In one embodiment of this aspect, AD101 may be administered at a daily dose (QD) of up to at least 180 mg. In a further embodiment of this aspect, the subject is also administered donepezil hydrochloride (e.g. including when AD101 is administered at a daily dose (QD) of up to at least 180 mg).

The disclosure also describes that AD101 may be given orally at a dose of up to at least 180 mg QD to subjects with Alzheimer's disease over a period of time longer than 12 weeks, e.g. 24 weeks or 36 weeks or longer, without inducing significant safety concerns in subjects, including when AD101 is co-administered with memantine hydrochloride and/or donepezil hydrochloride.

The disclosure further describes that AD101 administered at a dose of up to at least 180 mg QD to subjects with Alzheimer's disease, who are also being treated with memantine hydrochloride and/or donepezil hydrochloride (e.g. Aricept®), may be particularly effective to improve cognition and global function and/or delay decline in both of these outcomes in treated subjects.

Thus, in one aspect, the present disclosure provides a method of treating Alzheimer's disease in a human subject suffering therefrom comprising administering orally to a subject a daily dose of 180 mg of AD101.

In one aspect, the present disclosure provides a method of treating dementia of the Alzheimer's type in a human subject suffering therefrom comprising administering orally to a subject a daily dose of 180 mg of AD101.

In one aspect, the present disclosure provides a method of treating Alzheimer's disease in a human subject suffering therefrom comprising administering orally to a subject a daily dose of 180 mg of AD101 and a dose of donepezil hydrochloride.

In one aspect, the present disclosure provides a method of treating dementia of the Alzheimer's type in a human subject suffering therefrom comprising administering orally to a subject a daily dose of 180 mg of AD101 and a dose of donepezil hydrochloride.

In one embodiment of any of the aforementioned aspects, the subject is already treated with donepezil hydrochloride prior to the first administered dose of AD101.

In one embodiment of any of the aforementioned aspects, the subject is already treated with donepezil hydrochloride in a stable dose prior to the first administered dose of AD101.

In one aspect, the present disclosure provides a method of treating Alzheimer's disease in a human subject suffering therefrom comprising administering orally to a subject a therapeutically effective amount of AD101 and a therapeutically effective amount of memantine hydrochloride.

In one aspect, the present disclosure provides a method of treating Alzheimer's disease in a human subject suffering therefrom comprising administering orally to a subject a daily dose of 180 mg of AD101 and a therapeutically effective amount of memantine hydrochloride.

In one aspect, the present disclosure provides a method of treating dementia of the Alzheimer's type in a human subject suffering therefrom comprising administering orally to a subject a therapeutically effective amount of AD101 and a therapeutically effective amount of memantine hydrochloride.

In one aspect, the present disclosure provides a method of treating dementia of the Alzheimer's type in a human subject suffering therefrom comprising administering orally to a subject a daily dose of 180 mg of AD101 and a therapeutically effective amount of memantine hydrochloride.

In one embodiment of any of the aforementioned aspects, the subject is already treated with memantine hydrochloride prior to the first administered dose of AD101.

In one embodiment of any of the aforementioned aspects, the subject is already treated with memantine hydrochloride in a stable dose prior to the first administered dose of AD101.

In one embodiment of any of the aforementioned aspects, the subject is already treated with memantine hydrochloride and donepezil hydrochloride prior to the first administered dose of AD101.

In one embodiment of any of the aforementioned aspects, the subject is already treated with memantine hydrochloride and donepezil hydrochloride in stable doses prior to the first administered dose of AD101.

Donepezil hydrochloride (e.g. Aricept®) may conveniently be administered orally to the subject at a daily dose of about 5 mg to about 50 mg, including 5 mg, 10 mg or 23 mg, or via a once-per-week transdermal patch to give a daily dose of 5 mg or 10 mg. The course of administration of donepezil hydrochloride may be one or more months, e.g. at least 30 days. Subjects may be started on donepezil hydrochloride at a lower dose (e.g. 5 mg or 10 mg QD) later increased to a maintenance dose (e.g. 23 mg QD).

Memantine hydrochloride may conveniently be administered orally to the subject at a daily dose of about 5 mg to about 30 mg. In one embodiment, memantine hydrochloride is administered at a daily dose of 5 mg. In one embodiment, memantine hydrochloride is administered at a daily dose of 20 mg. In one embodiment, memantine hydrochloride is administered at an initial daily dose of 5 mg which is subsequently increased to a maintenance daily dose of 20 mg. In one embodiment, memantine hydrochloride is administered orally to the subject as an extended-release capsule at a daily dose of 7 mg, 14 mg or 28 mg. In one embodiment, memantine hydrochloride is initially administered as an extended-release capsule at a daily dose of 7 mg, which is subsequently increased to a maintenance daily dose of 14 mg or 28 mg.

### BRIEF DESCRIPTION OF THE FIGURES

FIG. 1 is a flowchart for a randomized, double-blind, placebo-controlled, Phase 3 clinical study of the efficacy, safety and tolerability of AD101 in the treatment of AD in subjects on stable treatment with donepezil hydrochloride. Primary, secondary and exploratory efficacy outcomes are monitored, together with safety and tolerability variables including treatment emergent signs and symptoms (TESS), treatment emergent abnormal laboratory values (TEAVs), serious adverse events (SAEs) and therapeutic drug monitoring (TDM).

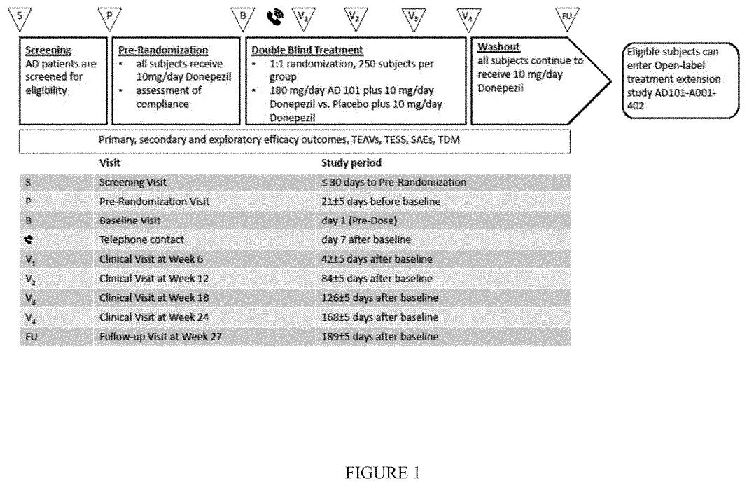

### DETAILED DESCRIPTION

AD101 has shown pharmacological activity in rodent models of learning and memory relevant to AD after both acute and chronic administration. AD101 has also been shown to increase acetylcholine (ACh) levels in rodent brains and to improve learning and memory in a number of behavioral tests in animals. (Yamaguchi Y., et al., J. Pharmacol. Exp. Ther. 577: 1079-1087 (2006); Ito Y., et ai, J. Pharmacol. Exp. Ther. 520: 819-827 (2007)). This functional improvement was correlated with enhancement in long-term potentiation (LTP), the electrophysiological correlate of memory formation, as well as with biochemical changes that are associated with enhanced LTP, such as increased activity of protein kinase C and Ca²⁺/calmodulin-dependent protein kinase II (CaMK II) (Han, F., et al., J. Pharmacol. Exp. Ther. 326: 127-134 (2008)).

Further experiments have shown that AD101 potentiates nicotine-stimulated release of ACh, increases extracellular ACh concentrations in the cerebral cortex, and increases extracellular concentrations of both ACh and dopamine in the hippocampus. The breadth of models across which AD101 exerts its effects suggests the potential for involvement at an upstream target in the signaling pathway(s) associated with these processes.

AD101 also decreases accumulation of Aβ-like deposits and produces an improvement in learning and memory functions, suggesting the behavioral effect of AD101 may be linked to reduction of Aβ production and/or accumulation (see US Patent Application Publication No. 2008/103158). AD101 has also been shown to induce cleavage of amyloid precursor protein, to decrease the level of pro-ADAM10 and/or BACE protein, and to enhance the activity of the ubiquitin-proteasome system pathway (see US Patent Application Publication Nos. 2010/0168135, 2010/0267763, and 2010/0298348). The contents of each of US Patent Application Publication Nos. 2008/103158, 2010/0168135, 2010/0267763, and 2010/0298348 are incorporated herein by reference.

Therefore, AD101 differs from marketed therapies in that it demonstrates two actions in animal research testing. It improves cognition and it also reduces the accumulation of abnormal protein deposits in the brain. These two properties suggest that AD101 is a promising agent for the treatment of AD.

Proof of concept trials in humans have investigated the safety and tolerability of AD101 over various doses, and its ability to improve cognition and global function during 12 weeks of administration. In one such trial, AD101 was administered at doses of 10 mg, 60 mg and 120 mg per day over 12 weeks to subjects aged 50 years or older having probable AD [defined by the fourth edition of the Diagnostic and Statistical Manual of Mental Disorders (DSM-IV) and the National Institute of Neurological and Communicative Disorders and Stroke/Alzheimer's Disease and Related Disorders Association criteria], a MMSE score of from 10 to 20, and a CT or MRI scan consistent with AD within 18 months of enrollment. Patients were required to be on a stable regimen of 10 mg of donepezil hydrochloride QD for at least 90 days prior to treatment with AD101, and continued treatment with 10 mg of donepezil hydrochloride QD during the trial with AD101.

The topline results of this trial were reported by S. Gauthier et al. in J Alzheimer's Dis. 2015; 48(2): 473-481. No significant AD101-related safety concerns were identified during the trial up to a dose of 120 mg QD, and the efficacy results supported the possibility that AD101, in patients receiving a stable dose of donepezil hydrochloride, may provide additional symptomatic benefit in moderate AD. Importantly, there was no discernable dose response across the treatment group in the primary outcome measures when the three doses were analyzed separately, and no indication that further increasing the dose of AD101 would lead to an additional improvement in treatment outcomes in the group of subjects or would not introduce safety issues. However, surprisingly, it has now been found that AD101 can be safely administered to AD subjects at daily doses up to at least 180 mg, and administering a daily dose of 180 mg AD101 to subjects with DAT, who are also treated with Aricept® (donepezil hydrochloride), can provide particularly effective symptomatic relief of AD without introducing significant AD101-related safety concerns.

The inventors of the present disclosure have thus identified a daily dose of AD101 which may be particularly effective to treat subjects with Alzheimer's Disease (AD), including subjects who are currently receiving a stable regimen of donepezil hydrochloride. AD101 administered orally at 180 mg QD may provide particular symptomatic relief to such subjects, including mitigation of cognitive impairment and global function in patients showing onset or development of Alzheimer's disease and/or improving cognition and global function in treated subjects.

The inventors of the present disclosure have also identified a new combination therapy which may be particularly effective to treat subjects with AD. Thus, the co-administration of AD101 and memantine hydrochloride may provide particular symptomatic relief to such subjects (including subjects who are currently receiving a stable regimen of donepezil hydrochloride), including mitigation of cognitive impairment and global function in patients showing onset or development of Alzheimer's disease and/or improving cognition and global function in treated subjects. The combination therapy may be effective without concomitant significant safety issues. In one embodiment of this aspect, AD101 may conveniently be administered orally to the subject at a daily dose of about 50 mg to about 250 mg, e.g. 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg or 250 mg. In one specific embodiment, AD101 is administered orally at a daily dose of about 120 mg. In one specific embodiment, AD101 is administered orally at a daily dose of about 180 mg.

A stable regimen of donepezil hydrochloride and/or memantine hydrochloride means herein a daily dose of donepezil hydrochloride and/or memantine hydrochloride that has been administered to treat a subject with Alzheimer's disease over an extended period of time, usually at least about 1, 2, 3, 4, 5 or 6 months or more (e.g. at least about 30 days), before the subject receives a first dose of AD101. Routinely, the subject continues to be administered donepezil hydrochloride and/or memantine hydrochloride during AD101 therapy.

In common practice, a subject may be treated with a daily dose of 5 mg donepezil hydrochloride administered as a single tablet, or 10 mg donepezil hydrochloride administered as one or two tablets, for the duration of treatment (e.g. 5 mg or 10 mg donepezil hydrochloride administered QD), or 10 mg donepezil hydrochloride administered daily as one or two tablets (e.g. 10 mg donepezil hydrochloride administered QD) for at least 30 days and then switched to a higher daily dose of 23 mg donepezil hydrochloride administered as a single tablet. Subjects administered a first dose of 180 mg AD101 may therefore also receive either 5 mg QD donepezil hydrochloride, 10 mg QD donepezil hydrochloride or 23 mg QD donepezil hydrochloride. Subjects with more severe AD may have been administered donepezil hydrochloride for a longer period of time, and may therefore be more likely to be receiving 23 mg QD donepezil hydrochloride when a first dose of 180 mg AD101 is administered.

Alternatively, donepezil hydrochloride may be administered via a transdermal patch, which may be replaced, for example, on a weekly basis. Such patches may conveniently deliver a daily dose 5 mg or 10 mg of donepezil hydrochloride. Examples of transdermal patches for use herein include ADLARITY®, which is a rectangular 6-layer laminate containing a tan colored overlay backing/adhesive layer without donepezil, separating layer, drug matrix, membrane, contact adhesive, and a release liner, and uses CORPLEX technology.

When a subject is treated with AD101 and memantine hydrochloride, the subject may conveniently be given 5 mg memantine hydrochloride for the duration of the combination therapy. Alternatively, a subject is initially treated with 5 mg memantine hydrochloride, which is subsequently increased (e.g. after 3 months or more) to a maintenance daily dose of 20 mg, usually prior to first dosing with AD101. In one embodiment, memantine hydrochloride is administered orally to the subject as an extended-release capsule at a daily dose of 7 mg for the duration of the combination therapy. In a further embodiment, memantine hydrochloride is administered orally to the subject as an extended-release capsule at a daily dose of 14 mg for the duration of the combination therapy. In another embodiment, memantine hydrochloride is initially administered as an extended-release capsule at a daily dose of 7 mg, which is subsequently increased to a maintenance daily dose of 14 mg or 28 mg, usually prior to first dosing with AD101. In one embodiment, a subject may also conveniently be treated with a daily dose of 10 mg donepezil hydrochloride administered as one or two tablets for the duration of the combination therapy, or 10 mg QD donepezil hydrochloride for 3 months or more and then switched to a higher daily dose of 23 mg donepezil hydrochloride administered as a single tablet, usually prior to first dosing with AD101.

Subjects first administered AD101 may already be taking memantine hydrochloride at the lower dose or the higher maintenance dose. Similarly, subjects first administered AD101 may already be taking donepezil hydrochloride at the lower dose or the higher maintenance dose.

Subjects with more severe AD may have been administered memantine hydrochloride and/or donepezil hydrochloride for a longer period of time, and may therefore be more likely to be receiving the maintenance dose of memantine hydrochloride and/or the maintenance doses of donepezil hydrochloride when AD101 is first administered.

When memantine hydrochloride and donepezil hydrochloride are both administered to a subject with AD, they may be given as separate oral compositions or in a single oral composition. In one aspect, memantine hydrochloride and donepezil hydrochloride are administered together as a capsule comprising memantine hydrochloride extended-release (14 mg or 28 mg) and 10 mg donepezil hydrochloride.

Namenda® (memantine hydrochloride) is commercially available and supplied as capsule-shaped, film-coated tablets containing 5 mg or 10 mg memantine hydrochloride or as a 2 mg/mL oral solution. Namenda® XR (memantine hydrochloride) is commercially available and supplied as extended-release capsules containing 7 mg, 14 mg, 21 mg or 28 mg memantine hydrochloride.

Aricept® (donepezil hydrochloride) is commercially available and supplied as film-coated round tablets containing 5 mg, 10 mg or 23 mg of donepezil hydrochloride.

AD101 may be administered orally as tablets or capsules which can contain from about 0.01% to about 99%, or from about 0.25% to about 75% of the active ingredient, together with one or more excipients or carriers.

Although AD101 may be combined with memantine hydrochloride and/or donepezil hydrochloride and used in a single oral dosage form, conveniently AD101, memantine hydrochloride and donepezil hydrochloride are administered in separate oral dosage forms.

A common test for assessing the severity of a subject's AD, or related dementia, is the Mini-Mental State Examination (MMSE). MMSE is used to measure thinking ability (or “cognitive impairment”). It assesses six items: orientation, learning, attention, word recall, language use and comprehension, and constructional praxis. Higher scores indicating greater cognitive function. MMSE has a maximum score of 30 points. The scores are generally grouped as follows:
25-30 points: normal cognition
21-24 points: mild dementia
10-20 points: moderate dementia
9 points or lower: severe dementia

In the present disclosure, a subject with Alzheimer's disease is one with an MMSE score up to 24, and who may, for example, have an MMSE score between 10 and 24 at the initiation of AD101 treatment.

Pharmaceutical compositions comprising AD101 may conveniently be obtained by combining AD101 with solid excipients/carriers, with optional grinding of the mixture and further processing using standard procedures well known to the skilled artisan to produce tablets or capsules. Suitable excipients include, for example: fillers, such as saccharides, e.g. lactose or sucrose, mannitol or sorbitol; cellulose preparations (e.g. microcrystalline cellulose) and/or calcium phosphates, e.g. tricalcium phosphate or calcium hydrogen phosphate; as well as binders, such as starch paste, using, e.g. maize starch, wheat starch, rice starch, potato starch, gelatin, tragacanth, methyl cellulose, hydroxypropylmethyl cellulose (HPMC), hydroxypropyl cellulose (HPC), sodium carboxymethylcellulose, and/or polyvinyl pyrrolidone. If desired, disintegrating agents may be added, such as the above-mentioned starches and celluloses (including low-substituted, HPC such as LH-31) and also carboxymethyl-starch, cross-linked polyvinyl pyrrolidone, agar, or alginic acid or a salt thereof, such as sodium alginate. Flow-regulating agents and lubricants, e.g. silica, talc, stearic acid or salts thereof, such as magnesium stearate or calcium stearate, and or polyethylene glycol may also be utilized. Tablets may be coated, e.g. using suitable cellulose preparations, such as acetylcellulose phthalate or hydroxypropylmethyl cellulose phthalate. Dye stuffs or pigments may be added to the tablets.

Other pharmaceutical preparations, which can be used orally, include push-fit capsules made of gelatin, as well as soft, sealed capsules made of gelatin and a plasticizer, such as glycerol or sorbitol. The push-fit capsules can contain the active ingredient in the form of granules, which may be mixed with fillers, such as lactose, binders such as starches, and/or lubricants such as talc or magnesium stearate and, optionally, stabilizers. In soft capsules, the active ingredient can be dissolved or suspended in suitable liquids, such as fatty oils, or liquid paraffin. In addition, stabilizers may be added.

Also included herein are dosage forms of AD101, in which the oral pharmaceutical preparations comprise an enteric coating. The term “enteric coating” is used herein to refer to any coating over an oral pharmaceutical dosage form that inhibits dissolution of the compound in acidic media, but dissolves rapidly in neutral to alkaline media and has good stability to long-term storage. Alternatively, the dosage form having an enteric coating may also comprise a water-soluble separating layer between the enteric coating and the core. The core of the enterically coated dosage form comprises AD101. Optionally, the core also comprises pharmaceutical additives and/or excipients. The separating layer may be a water-soluble inert active ingredient or polymer for film coating applications. The separating layer is applied over the core by any conventional coating technique known to one of ordinary skill in the art. Examples of separating layers include, but are not limited to sugars, polyethylene glycol, polyvinylpyrrolidone, polyvinyl alcohol, hydroxypropyl cellulose, polyvinylacetal diethylaminoacetate and hydroxypropylmethyl cellulose. The enteric coating is applied over the separating layer by any conventional coating technique. Examples of enteric coatings include, but are not limited to cellulose acetate phthalate, hydroxypropyl methylcellulose phthalate, polyvinyl acetate phthalate, carboxymethyl ethyl cellulose, copolymers of methacrylic acid and methacrylic acid methyl esters, such as Eudragit®L 12,5 or Eudragit®L 100 (Rohm Pharma), water-based dispersions such as Aquateric® (FMC Corporation), Eudragit®L 100-55. (Rohm Pharma) and Coating CE 5142 (BASF), and those containing water soluble plasticizers such as Citroflex® (Pfizer). The final dosage form is an enteric coated tablet, capsule or pellet. AD101 may conveniently be supplied for use as oval film-coated tablets containing 180 mg AD101.

AD101 may be administered in suitable dosage units (e.g. individual tablets), such as 10 mg, 30 mg, 60 mg, 90 mg or 180 mg units/tablets. Alternatively, larger dosage units may be provided, including, for example, units of about 200 mg to about 360 mg and all mg dose units therebetween, which may, if appropriate, be scored such that the unit can be readily broken into smaller units for administration. One or more such units may be administered to the subject to achieve the desired daily dose of 180 mg, e.g. 6×30 mg, 3×60 mg, 2×90 mg or 1×180 mg. When given as tablets, the tablet shape may conveniently be round, oblong or oval.

It will be appreciated that individual small units of drug, such as mini-tablets, may be combined to provide a unit suitable for administration as a daily dose of 180 mg. Such small units (e.g. mini-tablets) may be combined to produce, for example, a 10 mg, 30 mg, 60 mg, 90 mg or 180 mg unit (e.g. capsule).

Examples of 10 mg, 30 mg, 60 mg, 90 mg and 180 mg tablets are presented in Table 1 below:

TABLE 1

| | Quantity | Quantity | Quantity | Quantity | Quantity | |
|---|---|---|---|---|---|---|
| Component | (mg) | (mg) | (mg) | (mg) | (mg) | Function |
| AD101 | 10.0 | 30.0 | 60.0 | 90.0 | 180.0 | API |
| Mannitol* | 148.8 | 128.8 | 98.8 | 68.8 | N/A | Filler |
| Microcrystalline | 29.0 | 29.0 | 29.0 | 29.0 | 29.0 | Filler |
| cellulose** | | | | | | |
| Low-substituted | 10.5 | 10.5 | 10.5 | 10.5 | 10.5 | Disintegrator |
| HPC*** | | | | | | |
| HPC**** | 7.5 | 7.5 | 7.5 | 7.5 | 7.5 | Binder |
| Magnesium | 4.2 | 4.2 | 4.2 | 4.2 | 4.2 | Lubricant |
| stearate | | | | | | |
| Purified | N/A | N/A | N/A | N/A | N/A | N/A |
| water***** | | | | | | |
| Total | 210.0 | 210.0 | 210.0 | 210.0 | 231.2 | — |

*D-Pearlitol 160C;
**Avicel PH101;
***LH-31;
****HPC-SL;
*****Removed by diving during processing

In one aspect, 180 mg AD101 is administered QD each morning. In one aspect, 5 mg, 10 mg or 23 mg donepezil hydrochloride is also administered each day (e.g. 5 mg QD, 10 mg QD or 23 mg QD). The concomitant administration of 180 mg QD AD101 and 5 mg, 10 mg or 23 mg donepezil hydrochloride (e.g. 5 mg QD, 10 mg QD or 23 mg QD) may be continued until either unacceptable safety issues appear or the subject no longer receives benefit from the combination of drugs. In practice, AD101 and donepezil hydrochloride should be administered daily without interruption for as long as the subject receives benefit. Certain subjects receiving AD101 and donepezil hydrochloride as described above may also be receiving a suitable dose of memantine hydrochloride.

In one aspect, subjects who may particularly benefit from treatment with 180 mg QD AD101 include individuals diagnosed with probable AD (e.g. as defined by the National Institute of Neurological and Communicative Disorders and Stroke—Alzheimer's Disease and Related Disorders Association).

In one aspect, subjects who may particularly benefit from treatment with 180 mg QD AD101 include individuals diagnosed with dementia due to Alzheimer's Disease (e.g. consistent with NIA/AA criteria) and an absence of vascular cognitive disorders (e.g. as defined by 2014 VASCOG criteria or a modified Hachinski score of >4).

One or more additional therapeutic agents may be administered with AD101, memantine hydrochloride and/or donepezil hydrochloride according to the present disclosure. Such agents may include therapeutic agents useful for treating subjects with Alzheimer's disease. When present, each active ingredient is conveniently administered as a separate composition.

During therapy, the effectiveness of treatment with 180 mg QD AD101 may be measured at different timepoints using, for example, the Alzheimer's Disease Assessment Scale, cognitive subscale (ADAS-Cog) and the Alzheimer Disease Cooperative Study-Clinical Global Impression Plus version (ADCS-CGI) for global function, or any other measure of subject function at the discretion of the subject's medical care providers, including, but not limited to, MMSE for cognition, the Clinical Dementia Rating Sum of Boxes (CDR-SB) for global function and the Neuropsychiatric Inventory (NPI) for behavior symptoms. Changes in appearance and presence of certain biomarkers, such as longitudinal changes in plasma concentrations of phospho-tau217, beta amyloid 42 and 4 may also be measured.

Example 1 hereinafter is a safety extension study, in which subjects who completed an earlier AD101 monotherapy AD trial or an earlier AD101+Aricept® AD trial were subjected to a 3 month dose escalation trial in which the AD101 dose was increased from 60 mg QD to 180 mg QD. 5 subjects from the AD101+Aricept® AD trial who were also receiving memantine hydrochloride were enrolled in the extension study.

### Example 1

### Safety Extension Study

A 12-week, open-label, multicenter, safety extension study was conducted on AD subjects who completed an earlier preliminary efficacy and safety study of AD101 in AD (Clinicaltrials.gov identifier: NCT00842673; ST101-A001-201) and AD subjects who completed an earlier preliminary efficacy and safety study of AD101 plus Aricept® in AD (Clinicaltrials.gov identifier: NCT00842816; ST101-A001-202). Subjects were titrated as follows: all subjects receive 60 mg once daily for the first month, 120 mg once daily for the second month and 180 mg once daily for the third month. Dose increases were dependent on the subject's tolerability of the previous dose. The purpose of this study was to assess safety and tolerability in this population with additional exposure to AD101.

### Results

Table 2 summarizes subject disposition. A total of 293 AD subjects were enrolled (126 subjects from the ST101-A001-201 study [89% eligible subjects] and 167 subjects from the ST101-A001-202 study [90% eligible subjects]). 257 (87.7%) subjects completed the extended study. Greater than 95% of subjects eligible to increase their dose to 180 mg did so at the Week 8 visit. Discontinuations were similar for each dose level with 5.5% (16 subjects), 4.8% (14 subjects), and 2.0% (6 subjects) discontinuing the study at the 60 mg, 120 mg, and 180 mg dose levels, respectively.

TABLE 2

| | 60 mg | | 120 mg | | 180 mg | | Total | |
|---|---|---|---|---|---|---|---|---|
| | (N = 293) | | (N = 276) | | (N = 243) | | (N = 293) | |
| | n (%) | | n (%) | | n (%) | | n (%) | |
| Completed Study | 257 | (87.7) | 256 | (87.4) | 237 | (80.9) | 257 | (87.7) |
| Discontinued Early | 16 | (5.5) | 14 | (4.8) | 6 | (2.0) | 36 | (12.3) |
| Subject's Request | 5 | (1.7) | 6 | (2.0) | 1 | (0.3) | 12 | (4.1) |
| Protocol Violation | 1 | (0.3) | 1 | (0.3) | 0 | | 2 | (0.7) |
| Adverse Event | 8 | (2.7) | 4 | (1.4) | 5 | (1.7) | 17 | (5.8) |

| | | | | | | | |
|---|---|---|---|---|---|---|---|
| Other | 2 | (0.7) | 3 | (1.0) | 0 | 5 | (1.7) |

Notes:
Subjects can be in more than one AD101 treatment group. 60 mg (4 wks) → 120 mg (4 wks) → 180 mg (4 wks); administered QD for 3 months total; Dose escalation if subject and investigator agreed; total column counts unique subjects; the denominator is 293 (total enrolled) for all percentages.

### Overall, Safety and Tolerability

The safety population for this study is comprised of 293 subjects.

There were 17 Serious Adverse Events (SAEs) that occurred in 17 subjects (1 death due to multiple cerebral vascular accidents, deemed unrelated, that occurred 10 days post-study after subject received 60 mg, 120 mg, and 180 mg AD101 for 4 weeks each; 7 on 60 mg AD101, 5 on 120 mg AD101, and 4 on 180 mg AD101), summarized in Table 3. Eleven of the seventeen subjects discontinued, and six subjects completed this study. All SAEs were considered by the investigator to be unrelated to study drug with the exception of SAE #00036 (Syncope in an 86 year old female on 180 mg ST101).

TABLE 3

| SAE # | Age | Sex | Dose* | | Serious Criterion | Subject Status | Event |
|---|---|---|---|---|---|---|---|
| 00012 | 59 | M | N/A | | Hospitalization/ | Subject completed 201 | Multiple CVAs |
| | | | | | Death | and 401. Hospitalized | |
| | | | | | | with agitation 10 days | |
| | | | | | | after completing 401. | |
| | | | | | | Death occurred 4 days | |
| | | | | | | later. | |
| 00014 | 81 | F | 60 | mg | Hospitalization | Subject had an SAE in | Asthma |
| | | | | | | the 201 study; subject | exacerbation |
| | | | | | | was about 4 weeks into | |
| | | | | | | 401. Subject | |
| | | | | | | discontinued. | |
| 00017 | 78 | M | 60 | mg | Hospitalization | Subject was about 3 | Chest Pain |
| | | | | | | weeks into 401 when he | |
| | | | | | | suffered chest pain after | |
| | | | | | | shoveling snow. Subject | |
| | | | | | | completed 401. | |
| 00023 | 76 | F | 60 | mg | Hospitalization | Subject was 3 weeks into | Fractured Wrist |
| | | | | | | 401 when she was | from Accidental |
| | | | | | | tripped, fell and fractured | Fall |
| | | | | | | her wrist. Subject | |
| | | | | | | completed 401. | |
| 00025 | 81 | F | 60 | mg | Hospitalization | Subject was 4 weeks into | Seizure |
| | | | | | | the 401 study. | |
| | | | | | | Hypotension likely | |
| | | | | | | secondary to dehydration. | |
| | | | | | | Subject discontinued. | |
| 00027 | 79 | F | 60 | mg | Hospitalization | Subject was 2 weeks into | Syncope (vasovagal) |
| | | | | | | the 401 study. Subject | |
| | | | | | | completed 401. | |
| 00028 | 83 | M | 60 | mg | Hospitalization | Subject discontinued. | Vestibular neuronitis |
| 00043 | 78 | M | 60 | mg | Hospitalization | Subject was 3 weeks into | GI bleed 2°to |
| | | | | | | study when event | diverticular |
| | | | | | | occurred, discontinued. | disease |
| 00013 | 74 | M | 120 | mg | Hospitalization | Subject completed 201 | Lightheaded |
| | | | | | | and was about 8 weeks | (Sick sinus |
| | | | | | | into 401 when event | syndrome) |
| | | | | | | occurred Subject | |
| | | | | | | discontinued. | |
| 00019 | 77 | F | 120 | mg | Hospitalization | Subject was 5 weeks into | Fractured right |
| | | | | | | 401 when she tripped/fell | hip |
| | | | | | | while chasing her cat. | |
| | | | | | | Subject discontinued | |
| | | | | | | (nursing home placement) | |
| 00020 | 69 | F | 120 | mg | Hospitalization | Subject was ~8 weeks | Worsening of |
| | | | | | | into 401. Subject | Diverticulitis |
| | | | | | | discontinued. | |
| 00035 | 88 | M | 120 | mg | Hospitalization | Subject discontinued. | Exacerbation of |
| | | | | | | | abd. aortic |
| | | | | | | | aneurysm |
| 00044 | 83 | F | 120 | mg | Hospitalization | Event occurred during 5^{th} | Fx Left |
| 00045 | | | | | | week; subject completed | Humerus/Post- |
| | | | | | | 401. | op Hypoxia 2°to CHF |
| 00036 | 86 | F | 180 | mg | Hospitalization | Subject was evaluated for | Syncope |
| | | | | | | syncope several times in | |
| | | | | | | the past. Subject | |
| | | | | | | discontinued. Subject also | |
| | | | | | | had an SAE in the 201 | |
| | | | | | | study (UTI). | |
| 00011 | 88 | M | 180 | mg | Hospitalization | Subject tripped over his | Fractured |
| | | | | | | shoes and fell; | Vertebrae |
| | | | | | | discontinued. | |
| 00021 | 72 | M | 180 | mg | Hospitalization | Subject was ~9 weeks into | Chest Pain |
| | | | | | | 401 and had discontinued | |
| | | | | | | due to anxiety 2 days prior | |
| | | | | | | to the hospitalization. | |
| 00039 | 71 | M | 180 | mg | Hospitalization | Completed 401; event | Pneumonia/Pulmonary |
| 00040 | | | | | | occurred one day later. | Adenocarcinoma |

*= At the time of the event

Table 4 summarizes the number and percent of subjects with adverse events (AEs) by preferred term occurring in ≥2% of subjects. Adverse events were experienced by 51.2% of subjects of the 293 subjects that enrolled this study. The AEs were assigned to a dose group according to the dose the subject was on at the time the event occurred. Because all subjects received AD101, it is reasonable to compare the AEs reported in this study to the AEs that were considered commonly reported in the studies 201 and 202, which contributed the subjects for the present study. Of the 10 AEs that made the 2% cut this study, 3/10 did not make the 5% cut in 201 or 202 (agitation, fatigue, and contusion). There does not appear to be any pattern related to AD101 dose in regard to the nature or frequency of the specific AE.

TABLE 4

| | ST101 | | | | | | | |
|---|---|---|---|---|---|---|---|---|
| | Total | | 60 mg | | 120 mg | | 180 mg | |
| System Organ Class/ | (N = 293) | | (N = 293) | | (N = 276) | | (N = 243) | |
| Preferred Term | n (%) | | n (%) | | n (%) | | n (%) | |
| Subjects with any AE | 150 | (51.2) | 89 | (30.4) | 64 | (23.2) | 53 | (21.8) |
| Infections & Infestations | 40 | (13.7) | 20 | (6.8) | 11 | (4.0) | 10 | (4.1) |
| Urinary Tract Infection | 12 | (7.6) | 3 | (1.0) | 6 | (2.2) | 4 | (1.6) |
| Upper Respiratory Tract Infection | 7 | (2.4) | 3 | (1.0) | 2 | (0.7) | 2 | (0.8) |
| Nervous System Disorders | 33 | (11.3) | 15 | (5.1) | 11 | (4.0) | 8 | (3.3) |
| Dizziness | 8 | (2.7) | 4 | (1.4) | 4 | (1.4) | 1 | (0.4) |
| Somnolence | 7 | (2.4) | 5 | (1.7) | 2 | (0.7) | 0 | |
| Psychiatric Disorders | 26 | (8.9) | 12 | (4.1) | 8 | (2.9) | 7 | (2.9) |
| Agitation | 9 | (3.1) | 5 | (1.7) | 2 | (0.7) | 3 | (1.2) |
| General Disorders & Administration | 25 | (8.5) | 12 | (4.1) | 10 | (3.6) | 3 | (1.2) |
| Site Condition | | | | | | | | |
| Oedema Peripheral | 9 | (3.1) | 5 | (1.7) | 2 | (0.7) | 2 | (0.8) |
| Fatigue | 6 | (2.0) | 3 | (1.0) | 3 | (1.1) | 0 | |
| Gastrointestinal Disorders | 22 | (7.5) | 10 | (3.4) | 5 | (1.8) | 8 | (3.3) |
| Diarrhea | 8 | (2.7) | 6 | (2.0) | 2 | (0.7) | 1 | (0.4) |
| Injury, Poisoning and | 21 | (7.2) | 12 | (4.1) | 8 | (2.9) | 5 | (2.1) |
| Procedural Complications | | | | | | | | |
| Fall | 12 | (4.1) | 8 | (2.7) | 5 | (1.8) | 1 | (0.4) |
| Contusion | 6 | (2.0) | 3 | (1.0) | 3 | (1.1) | 1 | (0.4) |

Notes:
Subjects can be in more than one AD101 treatment group. Total column counts unique subjects. All AEs were treatment-emergent and coded using MedDRA 12.1.

### Subjects in the Safety Extension Study Started on Memantine Hydrochloride

There were 5 subjects from the ST101-A001-202 study who initiated memantine hydrochloride when enrolled in the open-label extension study (the 401 study). In all cases, the subjects completed the 401 study and dosed up to 180 mg AD101 as per protocol. Information on these 5 subjects, including adverse events, is summarized in Table 5. No clinically significant laboratory or vitals sign abnormalities were observed.

TABLE 5

| | | | Study Drug | Memantine | Adverse Events during |
|---|---|---|---|---|---|
| Subject # | Sex | Age | Dose (mg) | Dose(s) | Memantine Go-administration |
| 03-2003 | F | 81 | 202: PBO | 5 mg BID | No new AEs. |
| | | | 401: 180 | ongoing | |
| 18-2018 | F | 55 | 202: 10 | 10 mg QD | URI × 14 days began 30 days |
| | | | 401: 180 | | after memantine start, |
| | | | | | moderate, not related. |
| | | | | | Decreased appetite × 9 days |
| | | | | | began 36 days after memantine |
| | | | | | start, moderate, possibly |
| | | | | | related. |
| | | | | | UTI × 7 days began 30 days |
| | | | | | after memantine start, |
| | | | | | moderate, not related. |
| | | | | | Confusion × 8 days, began 38 |
| | | | | | days after memantine start, |
| | | | | | moderate, possibly related. |
| | | | | | Anxiety, ongoing at |
| | | | | | completion, began 50 days |
| | | | | | after memantine start, |
| | | | | | moderate, not related. |
| 14-2108 | F | 79 | 202: 10 | 5 mg QD −> | No new AEs. |
| | | | 401: 180 | 5 mg BID −> | |
| | | | | 15 mg QD −> | |
| | | | | 10 mg BID | |
| | | | | over 4 weeks | |
| 18-2122 | M | 76 | 202: 60 | 10 mg QD | No new AEs. |
| | | | 401: 180 | | |
| 14-2178 | M | 62 | 202: PBO | 5 mg QD −> | Sinus headache × 1 day, |
| | | | 401: 180 | 5 mg BID −> | occurred 1^{st} day of memantine, |
| | | | | 15 mg QD −> | mild, not related. |
| | | | | 10 mg BID | Dizzy/lightheaded over 18 |
| | | | | over 4 weeks | days, began 6 days after |
| | | | | | memantine titration to 10 mg |
| | | | | | QD complete mild, possibly |
| | | | | | related. |
| | | | | | Vomiting × 1 day, occurred 2 |
| | | | | | weeks after memantine dose |
| | | | | | titration to 10 mg BID was |
| | | | | | complete, moderate, not |
| | | | | | related. |

### CONCLUSIONS

Study 401 was an open-label, extension study that enrolled subjects who completed Studies 201 or 202. No new safety or tolerability issues were identified in this 3-month open-label safety extension study. Administration of AD101 at a dose of 180 mg QD was found to be at least as safe as administering the drug at lower doses of 60 mg QD and 120 mg QD. Furthermore, no clinically significant laboratory or vitals sign abnormalities were observed for the 5 subjects taking memantine hydrochloride.

All references, articles, publications, patents, patent publications, and patent applications cited herein are incorporated by reference in their entireties for all purposes. However, mention of any reference, article, publication, patent, patent publication, and patent application herein is not, and should not, be taken as acknowledgment or any form of suggestion that they constitute valid prior art or form part of the common general knowledge in any country in the world.

1. (canceled)

2. (canceled)

3. A method of treating Alzheimer's Disease comprising administering orally to a human subject a daily dose of 180 mg of AD101.

4. A method of treating Alzheimer's Disease comprising administering orally to a human subject a daily dose of 180 mg of AD101 and a dose of donepezil hydrochloride.

5. (canceled)

6. (canceled)

7. (canceled)

8. (canceled)

9. (canceled)

10. A method of treatment according to claim 4, where the subject is already treated with donepezil hydrochloride in a stable dose prior to the first administered dose of AD101.

11. A method of treatment according to claim 4, wherein donepezil hydrochloride is administered to the subject at a daily dose of 5 mg, 10 mg or 23 mg.

12. A method of treatment according to claim 11, wherein donepezil hydrochloride is administered orally (e.g. as a film-coated tablet or an orally disintegrating tablet).

13. (canceled)

14. (canceled)

15. (canceled)

16. (canceled)

17. (canceled)

18. A method of treatment according to claim 11, wherein donepezil hydrochloride is administered at an initial daily dose of 10 mg which is increased to a maintenance daily dose of 23 mg donepezil hydrochloride.

19. A method of treatment according to claim 3, wherein AD101 is administered as a one or more tablets once-daily (QD).

20. A method of treatment according to claim 19, wherein AD101 is administered as 3×60 mg, 2×90 mg or 1×180 mg tablets.

21. (canceled)

22. A method of treatment according to claim 3, wherein the subject is treated with one or more additional therapeutic agents.

23. (canceled)

24. A method of treatment according to claim 22, wherein AD101 and one or more additional therapeutic agents are each administered as separate compositions.

25. A method of treatment according to claim 4, wherein the co-administration of AD101 and donepezil hydrochloride has an additional beneficial effect on the condition of the treated subject relative to the administration of AD101 or donepezil hydrochloride alone.

26. A method of treatment according to claim 3, wherein the subject demonstrates an improvement in cognition or global function following treatment.

27. A method of treating Alzheimer's disease comprising administering orally to a human subject a therapeutically effective amount of AD101 and a therapeutically effective amount of memantine hydrochloride.

28. (canceled)

29. (canceled)

30. A method of treatment according to claim 27, where the subject is already treated with memantine hydrochloride in a stable dose prior to the first administered dose of AD101.

31. (canceled)

32. A method of treatment according to claim 27, wherein AD101 is administered at a daily dose of 180 mg.

33. A method of treatment according to claim 32, wherein AD101 is administered as one or more tablets once-daily (QD).

34. A method of treatment according to claim 33, wherein AD101 is administered as 3×60 mg, 2×90 mg or 1×180 mg tablets.

35. A method of treatment according to claim 27, wherein memantine hydrochloride is administered orally to the subject at a daily dose of about 5 mg to about 30 mg.

36. (canceled)

37. (canceled)

38. (canceled)

39. (canceled)

40. (canceled)

41. (canceled)

42. (canceled)

43. (canceled)

44. A method of treatment according to claim 32, wherein the subject is also treated with donepezil hydrochloride.

45. (canceled)

46. (canceled)

47. (canceled)

48. (canceled)

49. (canceled)

50. (canceled)

51. A method of treatment according to claim 32, wherein the co-administration of AD101 and memantine hydrochloride has a synergistic effect on the condition of the treated subject.

52. A method of treatment according to claim 44, wherein the co-administration of AD101, memantine hydrochloride and donepezil hydrochloride has a synergistic effect on the condition of the treated subject.

53. A method of treatment according to claim 44, wherein the subject demonstrates an improvement in cognition or global function following treatment.

54. A method of treatment according to claim 3, wherein the human subject has an MMSE score of 10 to 24 at the onset of treatment with AD101.

55. (canceled)

56. (canceled)

57. (canceled)

58. (canceled)

59. A method of treatment according to claim 44, wherein the human subject has an MMSE score of 10 to 24 at the onset of treatment with AD101.

60. (canceled)

61. (canceled)

62. (canceled)

63. (canceled)

64. A method of treatment according to claim 3, wherein the subject exhibits dementia of the Alzheimer's type.

65. (canceled)

66. (canceled)
